(19) United States
(12) Patent Application Publication
(10) Pub. No.: US 2004/0010138 A1
(43) Pub. Date: Jan. 15, 2004

(54) Process for the production of 7alpha-methyl steroids

(75) Inventors: Marc Willuhn, Berlin (DE); Orlin Petrov, Berlin (DE); Evelin Amoulong-Kirstein, Berlin (DE); Stephan Vettel, Berlin (DE); Agnes Matzeit, Unna (DE)

Correspondence Address: MILLEN, WHITE, ZELANO & BRANIGAN, P.C., 2200 CLARENDON BLVD., SUITE 1400, ARLINGTON, VA, 22201, US

(73) Assignee: Schering AG, Berlin (DE)

(21) Appl. No.: 10/393,399

(22) Filed: Mar. 21, 2003

Related U.S. Application Data
(60) Provisional application No. 60/378,127, filed on May 16, 2002.

(30) Foreign Application Priority Data
Mar. 21, 2002 (DE) 10213371.9

(51) Int. Cl. C07J 1/00; C07J 21/00

(52) U.S. Cl. 540/4; 540/38; 552/505; 552/619

(57) ABSTRACT

This invention relates to a process for the production of 7α-methyl steroids of general formula I,

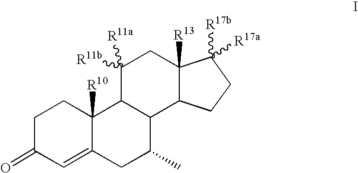

starting from compounds of general formula II,

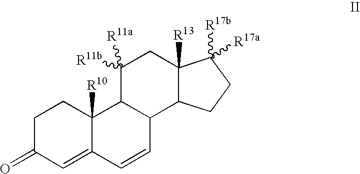

which are reacted in an aprotic solvent in the presence of 1-30 mol % of a copper compound CuYₙLₘ with 1-3 molar equivalents of CH₃MgX, then with a strong acid.

The process according to the invention is distinguished in that 7α-methyl steroids are obtained in good yields as well as high chemical purity and high diastereomer purities. The process is distinguished in that less waste accumulates with considerably higher throughput. The process according to the invention can therefore be suitable for the production of 7α-methyl steroids on the industrial scale.

This application claims the benefit of the filing date of U.S. Provisional Application Serial No. 60/377,127 filed May 16, 2002.

The invention relates to a process for the production of 7α-methyl steroids. The process according to the invention provides 7α-methyl steroids in good yields as well as high chemical purity and high diastereomer purities and allows the production of 7α-methyl steroids on an industrial scale.

Androgens, especially testosterone, are used for treating male menopause and for developing male sexual organs as well as for male birth control. In addition, these hormones also have partial anabolic active components that promote, i.a., muscle growth (E. Nieschlag, H. Behre, “Andrologie—Grundlagen und Klinik der reproduktiven Gesundheit des Mannes [Andrology—Principles and Clinical Studies of the Reproductive Health of the Man],” Springer Verlag, Berlin 2000).

For hormone replacement of testosterone in these indication areas, 7α-methyl-19-nortestosterone (17β-hydroxy-7α-methylestr-4-en-3-one) is proposed, which has, on the one hand, a higher biological action than testosterone (J. A. Campbell et al., Steroids 1963, 1, 317-324) since it has a higher binding affinity to the androgen receptor. On the other hand, it is distinguished by a metabolic stability that can presumably be attributed to a steric effect of the 7α-methyl group (WO 99/13812, WO 99/13883, U.S. Pat. No. 5,342,834; K. Sundaram et al., Int. J. Androl. 2000, 23 (Suppl. 2), 13-15; D. E. Cummings et al., J. Clin. Endocrinol. Metab. 1998, 83, 4212-4219).

It is common to most syntheses of 7α-methyl steroids that are known to one skilled in the art that the introduction of the 7α-methyl group is carried out by the 1,6-addition (S. Woodward, Chem. Soc. Rev. 2000, 29, 393-401) of an organometallic compound to a derivative of estra-4,6-dien-3-one.

In the process for the production of 7α-methyl-19-nortestosterone that is described in U.S. Pat. No. 4,000,273, 17β-acetoxy-4,6-estradien-3-one is reacted with at least three equivalents of dimethyllithium cuprate (Me₂CuLi), which is produced separately by reaction of methyllithium with a copper halide. Then, an acid work-up is done, the Δ^{4,5} is isomerized to the Δ^{3,4}-double bond, and the 17-acetyl group is saponified. After crystallization, 7α-methyl-19-nortestosterone is obtained in only 36% yield (Example 4 in U.S. Pat. No. 4,000,273). Drawbacks of this process are the low chemical yield, the low space/time yield, the high excesses of reagent and the high (hyperstoichiometric) quantity of copper salts. This results in addition in problems both in the removal of copper from the active ingredient, and, for environmental reasons, in waste water disposal.

According to the process that is described in U.S. Pat. No. 3,341,557, a 6-dehydrotestosterone (4,6-estradien-3-one) is reacted with an excess of at least five equivalents of methylmagnesium halide in the presence of copper salts. Then, it is reacted with pyridine/acetic anhydride and purified twice by chromatography. The yield for 17β-acetoxy-7α-methyl-19-nortestosterone according to this method is approximately 34% (8.5 molar equivalents of CH₃MgBr). For the subsequent saponification to 7α-methyl-19-nortestosterone with K₂CO₃ in MeOH under reflux, no yield is indicated (Examples 27-28 in U.S. Pat. No. 3,341,557). The large excesses of the reagent methylmagnesium halide are a drawback of this process. The increased formation of methylated by-products thus occurs, and the low purity of the intermediate product makes the two-fold chromatographic purification necessary. In addition, the 17-acetate is partially saponified by the large excess of methylmagnesium halide. For the chromatographic purification, the already saponified intermediate product must again be acetylated. Another drawback in addition to the large reagent excesses and the expensive working-up and purification is the high dilution in the reaction and thus the low space/time yield.

The process that is described in U.S. Pat. No. 3,341,557 was used in a similar form also for other steroidal 4,6-dien-3-ones (J. A. Campbell et al., J. Am. Chem. Soc. 1959, 81, 4069-4074). In the reaction of 3-oxo-17α-pregna-4,6-diene-21,17-carbolactone, only 22% of the corresponding 7α-methyl compound was obtained after chromatographic purification and recrystallization (N. W. Atwater et al., J. Org. Chem. 1961, 26, 3077-3083). Also, at other points, it was described in the literature that the copper-catalyzed reaction of steroidal 4,6-dien-3-ones with alkyl-magnesium halide provides the 7α-alkyl compound only in poor yields (J. R. Grunwell et al., Steroids 1976, 27, 759-771; G. C. Buzby et al., J. Med. Chem. 1966, 9, 782-784; N. P. van Vliet et al., Rec. Trav. Chim. Pays-Bas 1986, 105, 111-115). For 7α-methyl testosterone, a yield of only 40% was obtained according to this process (M. E. Wolff et al., J. Med. Chem. 1970, 13, 531-534).

Because of low yields and low space/time yields as well as high excesses of reagents, it was not previously possible with any of the processes available to one skilled in the art to produce 7α-methyl steroids on an industrial scale economically and with reasonable environmental impact.

The object of this invention is therefore to find an improved process for synthesis of 7α-methyl steroids that requires smaller quantities of reagents, provides higher yields and a higher throughput, as much as possible without requiring chromatographic purification, and is economical and ecological at the same time and thus is suitable for industrial-scale production.

The object is achieved by this invention.

The latter relates to a process for the production of 7α-methyl steroids of general formula I,

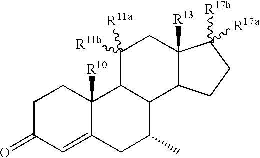

in which

R¹⁰ represents hydrogen or methyl,

R^{11a} represents hydrogen,

R^{11b} represents hydrogen, hydroxy, fluorine or —OC(O)R¹⁹, or together with R^{11a} represents an oxygen atom,

R¹⁹ represents a C₁-C₁₂-alkyl group,

R¹³ represents hydrogen, methyl or ethyl,

R^{17a} represents hydrogen,

R^{17b} represents hydrogen, hydroxy, R¹⁹, —OR¹⁹, —O(CO)R¹⁹, or together with R^{17a} represents an oxygen atom,

or

R^{17b} also can stand for the group —OM′,

in which

M′ represents —SiR¹R²R³,

R¹, R², R³, independently of one another, represent R¹⁹, —OR¹⁹, benzyl, aryl, or Oaryl,

which comprises the following steps:

a) Reaction of a compound of general formula II

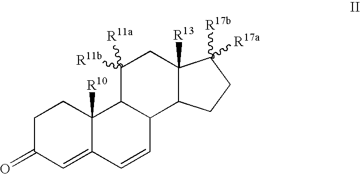

in which

R¹⁰ represents hydrogen or methyl,

R^{11a} represents hydrogen,

R^{11b} represents hydrogen, fluorine or —OC(O)R¹⁹, or together with R^{11a} represents an oxygen atom,

R¹⁹ represents a C₁-C₁₂-alkyl group,

R¹³ represents hydrogen, methyl or ethyl,

R^{17a} represents hydrogen,

R^{17b} represents hydrogen, hydroxy, R¹⁹, —OR¹⁹, —O(CO)R¹⁹, or together with R^{17a} represents an oxygen atom,

or

R^{17b} also can stand for the group —OM,

in which

M can represent -QR¹R²R³, or -QR¹R²,

Q can represent boron, aluminum, or silicon,

R¹, R², R³, independently of one another, can represent hydrogen, R¹⁹, —OR¹⁹, benzyl, aryl, or Oaryl,

in an aprotic solvent with 1-3 molar equivalents of CH₃MgX,

if

X represents chlorine, bromine, iodine or

with 0.5-3 molar equivalents of CH₃MgX,

if

X represents methyl,

in the presence of 1-30 mol % of a copper compound CuYₙLₘ,

in which

Y can be an inorganic or organic anion,

L can be a ligand,

n can be 1 or 2,

m can be 0 or a natural integer,

b) Addition of a strong acid to the reaction mixture and additional stirring,

c) Isolation and purification.

The C₁-C₁₂-alkyl groups for radical R¹⁹ can be, for example, unbranched alkyl groups such as the methyl, ethyl, n-propyl, n-butyl, n-pentyl, n-hexyl, n-heptyl, n-octyl, n-nonyl, n-decyl, n-undecyl or n-dodecyl group, or branched alkyl groups such as the iso-propyl, iso-butyl, sec-butyl, tert-butyl, iso-pentyl, neo-pentyl, 2-methylpentyl, 2,2-dimethylbutyl, 2,3-dimethylbutyl, 2-methylhexyl, 2,2-dimethylpentyl, 2,2,3-trimethylbutyl or 2,3,3-trimethylbutyl group.

The aryl groups for radicals R¹, R², and R³ can be, for example, phenyl, naphthyl, toloyl, xylyl, biphenyl, pyridyl and the corresponding substituted aryls.

The groups -QR¹R²R³, or -QR¹R² for radical OM, can therefore be, for example, trimethylsilyl, tert-butyldimethylsilyl, methyldiphenylsilyl, dimethylphenylsilyl, triethylsilyl, triisopropylsilyl, diphenylboryl, diethoxyboryl, triethoxyboryl, trimethoxyboryl, tri(tert-butoxy)aluminyl, triisopropoxyaluminyl, triethoxyaluminyl, or trimethoxyaluminyl.

The ligands can be, for example, the following compounds: water, dialkyl ethers such as diethyl ether, tetrahydrofuran, dialkyl sulfides such as dimethyl sulfide, diethyl sulfide or di(iso-propyl)sulfide; benzene; chiral or achiral phosphanes or bisphosphanes, such as, e.g., triphenylphosphanes, [1,1′-binaphthalene]-2,2′-diylbis(diphenylphosphane).

The inorganic anions can be, for example, fluoride, chloride, bromide, iodide or cyanide. The organic anions can be, for example, trifluoromethylsulfonate or acetate.

The copper compound CuYₙLₘ can be, for example, copper halides, such as copper(II) fluoride, copper(I) chloride, copper(II) chloride, copper(I) bromide, copper(I)-bromide-dimethyl sulfide complex, copper(II) bromide or copper(I) iodide, copper(I) cyanide, copper(I) trifluoromethylsulfonate, copper(II)trifluoromethylsulfonate, or copper(II) acetate.

The acids can be, for example, mineral acids such as sulfuric acid, phosphoric acid, hydrochloric acid, hydrobromic acid, hydroiodic acid or strong organic acids such as trifluoromethanesulfonic acid, methanesulfonic acid, or para-toluenesulfonic acid. Sulfuric acid is preferably used in step b).

The process according to the invention is suitable especially for the reaction of compounds of general formula II, in which R¹⁰ and R^{11a} represent hydrogen, R¹³ represents methyl, R^{11b} represents hydrogen or fluorine, R^{17b} represents OC(O)R¹⁹, R^{17a} represents hydrogen, or R^{17a} and R^{17b} together represent an oxygen atom, or R^{17b} represents —OM, to form compounds of general formula I.

For the implementation of the process according to the invention, for example, tetrahydrofuran (THF), 2-methyltetrahydrofuran or methyl-tert-butyl ether (MTBE) are suitable as aprotic solvents. The preferred solvent is tetrahydrofuran.

The quantity of solvent that can be used relative to the steroids of general formula II using the starting material (namely the dilution) can suitably be between 3-fold to 25-fold. The process according to the invention is distinguished, i.a., in that it also yields very good results in the low dilution ranges, with 3-fold to 10-fold solvent.

The reaction is performed at temperatures of −40° C. to 0° C. The preferred temperature range is between −35° C. to −15° C.

1.0-1.8 molar equivalents of CH₃MgX are preferably used in the presence of 5 to 25 mol % of copper compound.

If R^{17b} represents hydroxy or together with R^{17a} represents an oxygen atom, 2-2.8 equivalents of CH₃MgX are preferably used.

If X=methyl, instead of 1-3 molar equivalents of CH₃MgX, half of the quantity of (CH₃)₂Mg can also be used.

The reaction is especially preferably performed with 1.2-1.35 molar equivalents of methylmagnesium chloride in the presence of 10 to 20 mol % of copper(I) chloride. The especially preferred dilution is approximately 4 to 6× the volume of the solvent relative to the steroid that is used, and the especially preferred temperature range is between −35° C. to −15° C.

If necessary, an additional step can be performed between process steps b) and c) to remove the protective groups that are optionally present in the product, such as, e.g., —C(O)R¹⁹ or -M′.

The acyl groups C(O)R¹⁹ are removed by saponification with strong bases in alcoholic solvents to obtain the corresponding 11- or 17-hydroxy- or 11,17-dihydroxy-7α-methyl steroids. To this end, NaOH or KOH is preferably used in methanol, ethanol or iso-propanol. The protective group M′, which stands for a silyl group —SiR¹R²R³, can be removed, if necessary, according to standard methods that are known in the literature (for example, T. W. Greene, P. G. M. Wuts, Protective Groups in Organic Synthesis, 2^{nd} Edition, J. Wiley & Sons, New York 1991).

In addition, the optional additional step between process steps b) and c) of the reaction of compounds of general formula I, in which R^{17a} together with R^{17b} stands for an oxygen atom, can be used with suitable reducing agents to form the corresponding 17β-hydroxy derivatives. For this purpose, complex hydrides, such as, e.g., sodium borohydride, lithium borohydride, potassium borohydride, zinc borohydride, sodium triethoxy borohydride, sodium trimethoxy borohydride, lithium tri-(tert-butoxy)-aluminum hydride, lithium triisopropoxyaluminum hydride, lithium triethoxyaluminum hydride, and lithium trimethoxyaluminum hydride are preferably used.

The purification of the product is preferably carried out by crystallization from suitable solvents such as, e.g., methyl acetate, ethyl acetate, isopropyl acetate, MTBE, diethyl ether, di(iso-propyl)ether, THF, hexane, heptane, acetone, dichloromethane, toluene, methanol or ethanol. Especially good results are achieved with ethyl acetate or MTBE. The crystallization is performed at temperatures of −40° C. to reflux temperature, but preferably at −20° C. to 40° C.

When using common methods for synthesis of 17α-methyl-19-nortestosterones that are known to one skilled in the art, contamination is formed that inhibits the crystallization and thus reduces the yield of 7α-methyl steroid. According to the process of the invention, the formation of such by-products is also minimized. This circumstance results in the fact that the crystallization properties of the reaction products are advantageous such that the crystallization yield is increased. This is also ensured by the discontinuation of the 1,6-addition reaction being performed by quick addition of strong acids, and after the addition has been completed, stirring is continued for a certain time at pH values of less than 1. In most cases, 10-60 minutes is sufficient as additional stirring time.

The process according to the invention is distinguished in that 7α-methyl steroids are obtained in good yields as well as high chemical purity and high diastereomer purities. The process is distinguished in that less waste accumulates with considerably higher throughput. The process according to the invention can therefore be suitable for the production of 7α-methyl steroids on the industrial scale, especially for the production of 7α-methyl-19-nortestosterone, 17β-acetoxy-7α-methyl-19-nortestosterone, 7α-methyltestosterone, 17β-acetoxy-7α-methyltestosterone, 11β-fluoro-7α-methylestr-4-ene-3,17-dione, 11β-fluoro-17β-hydroxy-7α-methylestr-4-en-3-one, 7α-methylandrost-4-ene-3,11,17-trione, 17β-hydroxy-7α,18-dimethylestr-4-en-3-one, 17β-acetoxy-7α,18-dimethylestr-4-en-3-one, 17β-[(tert-butyldimethylsilyl)oxy]-7α-methylestr-4-en-3-one, and 17β-[(tert-butyldimethylsilyl)oxy]-11β-fluoro-7α-methylestr-4-en-3-one.

### Production Process

The starting materials of the synthesis according to general formula II can be produced in a way that is known in the art from the corresponding steroidal 4-en-3-ones, for example by conversion into the enol acetate followed by bromation and dehydrobromation (see J. Fried et al., Organic Reactions in Steroid Chemistry, Van Nostrand Reinhold, London 1972). Another possibility is the dehydrogenation of the corresponding steroidal 4-en-3-ones (see, e.g., E. J. Agnello et al., J. Am. Chem. Soc. 1960, 82, 4293-4299). Compounds of general formula II, in which R^{17b} stands for the group —OM are accessible by

a) Reaction of the compounds of general formula II, in which R^{17b} stands for a hydroxy group (—OH), with the reagents H-QR¹R²R³ of the hydride type, or with the reagents Cl-QR¹R²R³ or Cl-QR¹R² of the chloride type (see, for example, T. W. Greene, P. G. M. Wuts, Protective Groups in Organic Synthesis, 2^{nd} Edition, J. Wiley & Sons, New York 1991 or F. A. Carey, R. J. Sundberg, Organische Chemie [Organic Chemistry], VCH, Weinheim 1995);

b) Reaction of the compounds of general formula II, in which R^{17b} together with R^{17a} stands for an oxygen atom, with the reagents H-QR¹R²R³ of the hydride type (see, for example, F. A. Carey, R. J. Sundberg, Organische Chemie).

If necessary, protective groups can be cleaved according to the method that is known to one skilled in the art (T. W. Greene, P. G. M. Wuts, Protective Groups in Organic Synthesis).

Operating Instructions (AV 1) for the Production of 7α-Methyl Steroids:

140 ml of 3 M methylmagnesium chloride in THF (0.42 mol) is added in drops within 3-4 hours under nitrogen at −30° C. to a solution of 0.32 mol of a compound of general formula II and 5.7 g (0.058 mol) of copper(I) chloride in 500 ml of THF. Then, 32 ml of 50 vol. % sulfuric acid is added within 20 minutes and stirred for 30 more minutes. The mixture is diluted with 250 ml of water and stirred for 30 more minutes. The organic phase is separated and absorptively precipitated three times with 300 ml each of aqueous ammonium chloride/ammonia solution. The solvent is distilled off in a vacuum. Then, the product is optionally purified by filtration on silica gel and then crystallized from a suitable solvent.

If R^{17a} together with R^{17b} represents an oxygen atom, the compound of general formula II is reacted with double the quantity of methylmagnesium chloride.

If the product of the 1,6-addition to the cleavage of the acyl group —C(O)R¹⁹ is saponified, the reaction solution is further processed as follows:

The solution is concentrated by evaporation in a vacuum to about 400 ml. 100 ml of a 10% methanolic KOH solution is added to the solution and stirred under nitrogen for 3-4 hours. Then, a pH of 6 is set by adding 60 ml of 10% citric acid solution, and the solvent is distilled off in a vacuum. The residue is taken up in 1600 ml of MTBE, and the organic phase is washed with 400 ml of water. The product is crystallized by distilling off solvent in a vacuum and cooling to room temperature. The crystallizate is dissolved in 800-1200 ml of MTBE at 50-60° C., and the product is crystallized by distilling off to about 150-250 ml of residual volumes in the vacuum and cooling to room temperature.

### EXAMPLE 1

7α-Methyl-19-nortestosterone (17β-hydroxy-7α-methylestr-4-en-3-one)

According to AV 1, 56 g of 7α-methyl-19-nortestosterone (0.20 mol) is produced from 100 g of 17β-acetoxy-4,6-estradien-3-one (0.32 mol).

Yield: 62%

HPLC (100% purity): 99.1%

HRMS: Cld. 288.2089; Fnd. 288.2088

### EXAMPLE 2

7α-Methyltestosterone (17β-hydroxy-7α-methyl-androst-4-en-3-one)

Analogously to AV 1, 50 g of 7α-methyltestosterone (0.17 mol) is produced from 100 g of 17β-acetoxy-4,6-androstadien-3-one (0.30 mol).

Yield: 57%

HPLC (100% purity): 98.6%

HRMS: Cld. 302.2247; Fnd. 302.2245

### EXAMPLE 3

17β-Hydroxy-7α,18-dimethylestr-4-en-3-one

Analogously to AV 1, 53 g of 17β-hydroxy-7α,18-dimethylestr-4-en-3-one (0.18 mol) is produced from 100 g of 17β-acetoxy-18-methyl-4,6-estradien-3-one (0.30 mol).

Yield: 60%

HPLC (100% purity): 98.8%

HRMS: Cld. 302.2247; Fnd. 302.2246

### EXAMPLE 4

11β-Fluoro-7α-methylestr-4-ene-3,17-dione

Analogously to AV 1 with double the quantity of methylmagnesium chloride, 64 g of 11β-fluoro-7α-methylestr-4-ene-3,17-dione (0.21 mol) is obtained from 100 g of 11β-fluoroestra-4,6-diene-3,17-dione (0.35 mol).

Yield: 60%

HPLC (100% purity): 98.5%

HRMS: Cld. 304.1838; Fnd. 304.1838

### EXAMPLE 5

7α-Methylandrost-4-ene-3,11,17-trione

Analogously to AV 1 with double the quantity of methylmagnesium chloride, 58 g of 7α-methylandrost-4-ene-3,11,17-trione (0.19 mol) is obtained from 100 g of 4,6-androstadiene-3,11,17-trione (0.34 mol).

Yield: 56%

HPLC (100% purity): 99.0%

HRMS: Cld. 300.1725; Fnd. 300.1724

### EXAMPLE 6

17β-[(tert-Butyldimethylsilyl)oxy]-7α-methylestr-4-en-3-one

Analogously to AV 1, 15 g of 17β-[(tert-butyldimethylsilyl)oxy]-7α-methylestr-4-en-3-one (0.037 mol) is obtained from 20 g of 17β-[(tert-butyldimethylsilyl)oxy]-estra-4,6-dien-3-one (0.052 mol).

Yield: 71%

HPLC (purity 100%): 96.5%

HRMS: Cld. 402.2954; Fnd. 402.2950

The product of the reaction is then dissolved in 300 ml of acetone, mixed with 20 ml of 20% sulfuric acid, and stirred for 48 hours at room temperature. The reaction solution is extracted with MTBE, and the product is crystallized from MTBE (see Example 1).

Yield: 85%

HPLC (purity 100%): 97.6%

Operating Instructions (AV 2) for the Production of Compounds of General Formula II, in which R^{17b} Stands for the Group —OM

480 ml of a 1 M solution of lithium-tri-tert-butoxyaluminum hydride in THF is added in drops to a solution or suspension of 0.32 mol of a compound of general formula II, in which R^{17a} together with R^{17b} represent an oxygen atom, in 500 ml of THF, under nitrogen at 0° C., and stirring is continued for 30 minutes at 0° C.

The reaction solution is then further reacted according to AV 1.

### EXAMPLE 7

11β-Fluoro-17β-hydroxy-7α-methylestr-4-en-3-one

Analogously to AV 2, 59 g of 11β-fluoro-17β-hydroxy-7α-methylestr-4-en-3-one 0.19 mol) is obtained from 100 g of 11β-fluoroestra-4,6-diene-3,17-dione (0.35 mol).

Yield: 54%

HPLC (purity 100%): 97.3%

HRMS: Cld. 306.1995; Fnd. 306.1990

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

In the foregoing and in the examples, all temperatures are set forth uncorrected in degrees Celsius and, all parts and percentages are by weight, unless otherwise indicated.

The entire disclosures of all applications, patents and publications, cited herein and of corresponding German Application No. 102 13 371.9, filed Mar. 21, 2002, and U.S. Provisional Application Serial No. 60/378,127, filed May 16, 2002 are incorporated by reference herein.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. Process for the production of 7α-methyl steroids of general formula I, 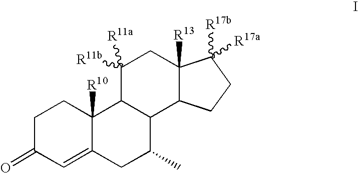
in which
R¹⁰ represents hydrogen or methyl,
R^{11a} represents hydrogen,
R^{11b} represents hydrogen, hydroxy, fluorine or —OC(O)R¹⁹, or together with R^{11a} represents an oxygen atom,
R¹⁹ represents a C₁-C₁₂-alkyl group,
R¹³ represents hydrogen, methyl or ethyl,
R^{17a} represents hydrogen,
R^{17b} represents hydrogen, hydroxy, R¹⁹, —OR¹⁹, —O(CO)R¹⁹, or together with R^{17a} represent an oxygen atom,
or
R^{17b} also can stand for the group —OM′,
in which
M′ represents —SiR¹R²R³,
R¹, R², R³, independently of one another, represent R¹⁹, —OR¹⁹, benzyl, aryl, or Oaryl,
comprising the following steps:
a) Reaction of a compound of general formula II 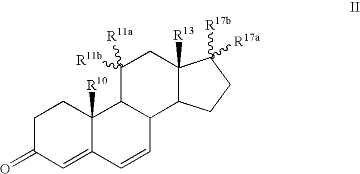
in which
R¹⁰ represents hydrogen or methyl,
R^{11a} represents hydrogen,
R^{11b} represents hydrogen, fluorine or —OC(O)R¹⁰, or together with R^{11a} represents an oxygen atom,
R¹⁹ represents a C₁-C₁₂-alkyl group,
R¹³ represents hydrogen, methyl or ethyl,
R^{17a} represents hydrogen,
R^{17b} represents hydrogen, hydroxy, R¹⁹, —OR¹⁹, —O(CO)R¹⁹, or together with R^{17a} represents an oxygen atom,
or
R^{17b} also can stand for the group —OM,
in which
M can represent -QR¹R²R³, or -QR¹R²,
Q can represent boron, aluminum, or silicon,
R¹, R², R³, independently of one another, can represent hydrogen, R¹⁹, —OR¹⁹, benzyl, aryl, or Oaryl,
in an aprotic solvent with 1-3 molar equivalents of CH₃MgX,
if
X can be chlorine, bromine, iodine or
with 0.5-3 molar equivalents of CH₃MgX,
if
X represents methyl,
in the presence of 1-30 mol % of a copper compound CuYₙLₘ,
in which
Y can be an inorganic or organic anion,
L can be a ligand,
n can be 1 or 2,
m can be 0 or a natural integer,
b) Addition of a strong mineral acid to the reaction mixture and additional stirring,
c) Isolation and purification.

2. Process according to claim 1, characterized in that sulfuric acid is used in step b).

3. Process according to one of the preceding claims, wherein in general formula II, R¹⁰ and R^{11a} represent hydrogen, R¹³ represents methyl, R^{11b} represents hydrogen or fluorine, R^{17b} represents OC(O)R¹⁹, R^{17a} represents hydrogen, or R^{17a} and R^{17b} together represent an oxygen atom, or R^{17b} represents —OM.

4. Process according to one of the preceding claims, wherein M in general formula II has the meaning of —AlR¹R²R³.

5. Process according to one of the preceding claims, wherein an additional step is performed between steps b) and c).

6. Process according to claim 5, wherein the additional step is the cleavage of the acyl group —C(O)R¹⁹ by saponification with a strong base in an alcoholic solvent.

7. Process according to claim 6, wherein NaOH or KOH is used as a base in methanol, ethanol or iso-propanol.

8. Process according to claim 5, wherein the additional step is the cleavage of group -M′.

9. Process according to claim 5, wherein the additional step is the reaction of compounds of general formula I, in which R^{17a} together with R^{17b} stand for an oxygen atom, with a reducing agent to form the corresponding 17β-hydroxy derivatives.

10. Process according to claim 9, wherein the reducing agent is a complex hydride reagent.

11. Process according to one of the preceding claims, wherein the purification is carried out by means of crystallization.

12. Process according to one of the preceding claims, wherein 1.0 to 1.8 molar equivalents of CH₃MgX are used in the presence of 5 to 25 mol % of a copper compound CuYₙLₘ.

13. Process according to one of the preceding claims, wherein 1.2 to 1.35 molar equivalents of methylmagnesium chloride in the presence of 10 to 20 mol % of copper(I) chloride.

14. Process according to one of the preceding claims, wherein the solvent is tetrahydrofuran.

15. Process according to one of the preceding claims, wherein the reaction is performed at a temperature of −35° C. to −15° C.

16. Process according to one of the preceding claims, wherein the reaction is implemented in a dilution of 3 to 10× the volume of solvent relative to the steroid that is used.

17. Process according to one of the preceding claims, wherein 1.2 to 1.35 molar equivalents of methylmagnesium chloride are used in the presence of 10 to 20 mol % of copper(I) chloride in tetrahydrofuran at a reaction temperature of −35° C. to −15° C. and in a dilution of 4 to 6× the volume of solvent relative to the steroid that is used.

18. Process according to claim 1, wherein in general formula II, R^{17b} represents hydroxy or together with R^{17a} represents an oxygen atom.

19. Process according to claim 18, wherein 2-2.8 molar equivalents of methylmagnesium chloride is used.

20. Process according to one of the preceding claims for the production of a compound from the group:
7α-Methyl-19-nortestosterone,
17β-acetoxy-7α-methyl-19-nortestosterone,
7α-methyltestosterone,
17β-acetoxy-7α-methyltestosterone,
11β-fluoro-7α-methylestr-4-ene-3,17-dione,
11β-fluoro-17β-hydroxy-7α-methylestr-4-en-3-one,
7α-methylandrost-4-ene-3,11,17-trione,
17β-hydroxy-7α,18-dimethylestr-4-en-3-one,
17β-acetoxy-7α,18-dimethylestr-4-en-3-one,
17β-[(tert-butyldimethylsilyl)oxy]-7α-methylestr-4-en-3-one,
17β-[(tert-butyldimethylsilyl)oxy]-11β-fluoro-7α-methylestr-4-en-3-one.
